(19) United States
(12) Patent Application Publication
(10) Pub. No.: US 2002/0156586 A1
(43) Pub. Date: Oct. 24, 2002

(54) Method for screening compounds

(75) Inventors: Albert Michiel van Rhee, Durham, NC (US); Kerry L. Spear, Raleigh, NC (US); P. Kay Wagoner, Chapel Hill, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ICAgen, Inc., Durham, NC

(21) Appl. No.: 10/077,358

(22) Filed: Feb. 15, 2002

Related U.S. Application Data
(60) Provisional application No. 60/270,365, filed on Feb. 20, 2001.

(51) Int. Cl. G06G 7/48; G06G 7/58; G06F 19/00; G01N 33/48; G01N 33/50

(52) U.S. Cl. 702/19; 703/11

(57) ABSTRACT

A method for screening compounds for biological activity is disclosed. The method may include selecting a test set of compounds and selecting a training set of compounds. An assay is performed on the training set of compounds and training set data are formed. This data are entered into a digital computer, and an analytical model is formed. A subset of compounds is identified using the analytical model.

### BACKGROUND OF THE INVENTION

In recent years, combinatorial chemistry coupled with high-throughput screening (HTS) has dramatically increased the number of compounds that are screened against biological targets. Despite the resulting explosion of screening data for a given target, hit rates still tend to be quite low (often less than 1%).

Current problems to be addressed in compound screening include the following: 1. the ability to increase the efficiency of primary screens, 2. the option to pursue multiple chemotypes in order to develop compounds along parallel product lines, and 3. the ability to explain nonlinear structure-activity relationships.

Various deterministic methods have been applied to address these problems. (Delaney, J. S., Mol. Diversity, 1:217-222 (1995); Bayley, M. J. et al., J. Mol. Graphics Modeling, 17:10-18 (1999); Wang, J. et al., J. Comb. Chem., 1:524-533 (1999); Klopman, G., J. Chem. Inf. Comput. Sci., 38:78-81 (1998); Burden, F. R., J. Chem. Inf. Comput. Sci., 38:47-53 (1998); Bemis, G. W. et al., J. Med. Chem., 39:2887-2893 (1996); Magalhães, N. S. S. et al., Eur. J. Med. Chem., 34:83-92 (1999); Chen, X. et al., J. Chem. Inf. Comput. Sci., 39:887-896 (1999)). The most limiting condition for these methods, however, is probably the requirement of the training set to encompass all chemotypes present in the test set, the so-called knowledge domain. Whereas this may not be of concern when discerning drug-like from nondrug-like compounds based on sufficiently diverse databases such as ACD (Available Chemical Directory. MDL Information Systems Inc., San Leandro, Calif.), CMC (Current Medicinal Chemistry. MDL Information Systems Inc., San Leandro, Calif.), or WDI (World Drug Index. Derwent Inc., Vienna, Va.), this becomes a self-limiting “conservatism in design” restriction (Coffen, D. L. et al., Med. Chem. Res., 8:206-218 (1998)) when screening for as yet unidentified activity in a narrowly defined chemical library.

QSAR (Quantitative Structure Activity Relationship) methods have been used to predict biological activity. However, the limitation of conventional QSAR methods is that a single (quasi-) linear equation is presumed to account for all biological activity. Whereas this may hold true for selective, reversible, and competitive binding models, these conditions need not necessarily apply to HTS data sets, especially HTS data sets for potential ion channel modulators (blockers, openers, or otherwise).

Ion channels are membrane embedded proteins of multimeric composition with intrinsic ion conduction properties. The intended pharmacological endpoint, i.e. activation, prolongation of activation, termination of activation, or block of the target ion channel, may be dependent on a number of factors including the site and mode of binding of a ligand to the channel. Past research (Holzgrabe, U. et al., Drug Disc. Today, 5:214-222 (1998); Zwart, R. et al., Mol. Pharmacol., 52:886-895 (1997); Chen, H. S. et al., J. Physiol., 499 (Pt 1):27-46 (1997)) indicates that it is very likely that chemical modulators of ion channels, especially those that are endogenously regulated by membrane potentials (e.g. the Kᵥ gene family) or ion concentrations (e.g., Ca²⁺ and Cl⁻ channels), are noncompetitive, or uncompetitive, allosteric modulators. An allosteric modulator is a compound that can bind to one site on a protein and can cause a conformational change in the protein such that the properties, e.g., activity, of another site of the protein are altered. Proteins modulated by allosteric modulators may have multiple binding sites, and compounds that interact with these multiple binding sites can alter the biological activity. It would be desirable if the analysis methods that are applied allow for the presence and/or selection of multiple binding mode models, rather than converge on a single unified model.

Embodiments of the invention address these and other problems.

### SUMMARY OF THE INVENTION

One embodiment of the invention is directed to a method for screening compounds for biological activity comprising: a) selecting a test set of compounds; b) selecting a training set of compounds; c) entering training set data into a digital computer, wherein the training set data are derived from a high throughput screening assay on the training set of compounds; d) forming an analytical model using a recursive partitioning process and the training set data; e) selecting a first subset of compounds using the analytical model; and f) selecting a second subset of compounds using a predetermined pharmaceutical or therapeutic profile.

One embodiment of the invention is directed to a method for screening compounds for biological activity, the method comprising: a) selecting a test set of compounds; b) selecting a training set of compounds; c) entering training set data into a digital computer, wherein the training set data are derived from a high throughput screening assay for ion channel modulators on the training set of compounds; d) forming an analytical model using the training set data and a recursive partitioning process; and e) identifying a subset of compounds using the analytical model. The ion channel modulators may be competitive or allosteric.

Another embodiment of the invention is directed to a computer readable medium comprising: a) code for entering training set data into a digital computer, wherein the training set data are derived from a high throughput screening assay for ion channel modulators (e.g., competitive or allosteric ion channel modulators) on the training set of compounds; b) code for forming an analytical model using the training set data and a recursive partitioning process; and c) code for identifying a subset of compounds from a test set of compounds using the analytical model.

Another embodiment is directed to a computer readable medium comprising: a) code for entering training set data into a digital computer, wherein the training set data are derived from a high throughput screening assay on the training set of compounds; b) code for forming an analytical model using a recursive partitioning process and the training set data; and c) code for selecting a subset of compounds using the analytical model; and d) code for selecting a subset of compounds according to a predetermined pharmaceutical or therapeutic profile.

These and other embodiments are described in further detail below.

### BRIEF DESCRIPTION OF THE DRAWINGS

FIG. 1 shows a flowchart illustrating a method according to an embodiment of the invention.

FIG. 2 shows a flowchart illustrating a process for forming an analytical model according to an embodiment of the invention.

FIG. 3 shows an example of a portion of a recursive partitioning tree.

FIG. 4 shows a graph of fold enrichment as a function of both the knot limit and the tree depth.

FIG. 5 shows a graph of % hit recovery as a function of both the knot limit and the tree depth.

FIG. 6 shows a graph of % hit recovery as a function of tree depth and fold enrichment. It shows the interdependency of optimization values. A Diverse Selection training set was used to form the graph.

FIGS. 7(a)-7(d) show graphs of % hit recovery as a function of both knot limit and tree depth, where each graph represents trees that are formed by different splitting processes. These figures show a comparison between splitting protocols.

FIG. 8(a) shows a graph showing the % hit recovery is plotted as a function of both the knot limit and the tree depth at an 80% threshold value applied to a DS data set.

FIG. 8(b) shows a graph showing the % hit recovery is plotted as a function of both the knot limit and the tree depth at an 85% threshold value applied to a DS data set.

FIG. 8(c) shows a graph showing the % hit recovery is plotted as a function of both the knot limit and the tree depth at an 80% threshold value applied to a RS data set.

FIG. 8(d) shows a graph showing the % hit recovery is plotted as a function of both the knot limit and the tree depth at a 90% threshold value applied to a DS data set. Collectively, FIGS. 8(a)-8(d) show comparisons between different types of training sets.

FIG. 9 shows the relative distribution of biological data as recorded by the HTS assay expressed by decile (squares), and a fitted Gaussian distribution function (dotted line). FIG. 9 shows the distribution of HTS data.

FIG. 10 shows a table showing the principal results from recursive partitioning models. It shows principal output measurements for each of the systematic variations in the training set, and the actualized measurements for the test set.

FIG. 11 shows a table showing the results per terminal node (Twoing-7-90). It shows the distribution of each of the compounds assigned to class 4 (highly active) with respect to their placement in terminal nodes I-VIII.

FIG. 12 shows a table showing the distribution of chemotypes per terminal node (Twoing-7-90). The relative distribution of each of the chemotypes (CT1-CT8) with respect to their occurrence in the terminal nodes I-VIII is shown.

FIG. 13 shows a table with HTS binning schemes. Compounds are assigned to one of four activity bins, depending on their biological activity as recorded by the HTS assay. Class 4 is considered “highly active”, class 3 “moderately active”, class 2 “weakly active”, and class 1 “inactive”. The distributions for three different thresholds are presented here.

### DETAILED DESCRIPTION

With the emergence of combinatorial chemistry, whether based on parallel, mixture, solution, or solid phase chemistry, large numbers of diverse or focused compound libraries can be generated. In embodiments of the invention, targeted libraries can be designed by applying non-parametric statistical processes such as recursive partitioning to large data sets containing thousands of compounds and their associated biological data.

Statistical processes including ANOVA, Hierarchical Cluster Analysis, Principal Components Analysis, Factor Analysis, Genetic Function Approximations, Partial Least Squares Fitting, multiple pharmacophore based models, and non-parametric statistical methods such as recursive partitioning can be used to screen compounds. Methodologically, parametric methods (Lucic, B. et al., J. Chem. Inf Comput. Sci., 39:121-132 (1999); Fujita, T. et al., J. Med. Chem., 10:991-1000 (1967); Hunt, P. A., J. Comput.-Aided Mol. Design, 13:453-467 (1999); Rogers, D. et al., J. Chem. Inf. Comput. Sci., 34:854-866 (1994); Dohoo, I. R. et al., Prev. Vet. Med., 29:221-239 (1997)) (i.e. a combination of chemical descriptors explains and predicts the biological activity of each compound in the training set) can be distinguished from nonparametric methods (i.e. one can calculate the chance of a compound being within a range of biological activities based on the distribution of chemical and biological descriptors in the training set).

The present inventors have determined that recursive partitioning can be used for predict which compounds are more likely than others to act as competitive or allosteric modulators of proteins. Embodiments of the invention preferably screen for allosteric modulators. The compounds that are screened may also be potential ion channel modulators. In preferred embodiments, the recursive partitioning process uses continuous range descriptors and multiple classes of biological activity to form analytical models. These models are especially predictive of biological activity and can be used to identify multiple chemotypes having high biological activity.

In embodiments of the invention, a recursive partitioning process is performed on a group of compounds. The group of compounds is recursively (i.e. starting with the complete set and ending with the smallest possible or allowable subset) split at a branch point into two statistically distinct nodes (subsets). Whereas variable selection in parametric methods is determined by their impact on correlation, recursive partitioning focuses on classification. As such, recursive partitioning has the possibility to optimize for synergism rather than additivity, for nonlinear relationships over forced (quasi-) linearity, and for multiple endpoints over single endpoints. In addition, during variable selection, recursive partitioning takes into account prior probabilities and penalties for misclassification. Recursive partitioning has diminishing numbers in each discriminant step. In contrast, parametric methods retain all information elements during the equation building phase.

Embodiments of the invention are particularly useful for screening compounds for use as ion channel modulators. Ion channel modulators are potentially useful for treating various disorders. Such modulators are useful for treating disorders, including CNS disorders, such as epilepsy and other seizure disorders, migraines, anxiety, psychotic disorders such as schizophrenia, bipolar disease, and depression. Such modulators are also useful as neuroprotective agents (e.g., to prevent stroke). Finally, such modulators could be useful for treating hypercontractility of muscles and cardiac arrhythmias, as analgesics, and as immunosuppressants or stimulants. In embodiments of the invention, modulators of multiple ion channel subtypes within ion channel families, so-called gene families, can be identified without focussing on a single binding site or mechanism.

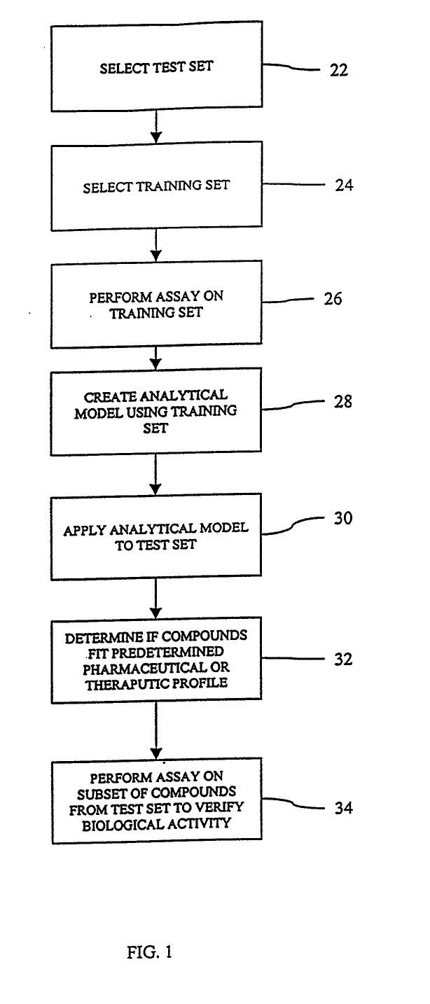

Referring to FIG. 1, one embodiment of the invention is directed to a method comprising selecting a test set of compounds (step 22) and selecting a training set of compounds (step 24). The test set and the training set may be selected from a test library. The test library may contain a number of compounds and characteristics for each of the compounds. For example, the test library may contain compounds and properties such as the molecular weight and the hydrophobic index of the compounds.

The compounds in the training set may be assayed using a high throughput screening assay to determine their biological activity (step 26). Once the biological activity data for the raining set is determined, an analytical model can be formed (step 28). A recursive partitioning process processes the raining set data to form the analytical model. The training set data may include compound information such as physicochemical properties of the compound (e.g., molecular weight) and the biological activity data for the compound (i.e., the degree of activity of the compound).

Using the analytical model, a first subset of compounds using the analytical method can be selected (step 30). A predetermined pharmaceutical or therapeutic profile may then be applied to the first subset to form a second subset of compounds (step 32). The predetermined pharmaceutical or therapeutic profile can be used to select compounds with a predetermined pharmaceutical or therapeutic goal in mind. For example, the goal may be to design a drug that dissolves in water. If a compound does not satisfy this profile, then it can be excluded from the second subset, thus reducing the number of possible candidates.

After forming the second subset of compounds, a second assay is performed on the second subset of compounds (step 34) to form a third subset of compounds. The second assay can be used to determine which of the second subset of compounds have the desired biological activity. The second assay may be of the same type as the first assay (performed on the training set) and can test for the biological activity of the compounds in the second subset.

Surprisingly and unexpectedly, embodiments of the invention can improve the hit rate of primary screens by at least 3-fold, while increasing screening efficiency. The improved hit rate can even be higher than 10- or 30-fold in embodiments of the invention. In some embodiments of the invention, less than ⅕^{th} of the complete selection (e.g., a test library) needs to be screened in order to identify about 75% of all actives present. Preferably less than 1/10^{th} of the complete selection needs to be screened in order to identify over 85% of all actives present.

I. Test Library

A test library of compounds may be identified. In some embodiments, the test library has a high information content (i.e., it can be maximally diverse within the relevant pharmaceutical and/or therapeutic diversity space). The test library may contain any suitable type of compound and any suitable information that is related to the compounds. For example, the compounds in the test library may be chemical compounds or biological compounds such as polypeptides. The test library may contain data relating to the compounds in the test library. For example, each compound in the test library may have chemical data such as a hydrophobic index and a molecular weight associated with it. The test library including the compounds and the information related to the compounds may be stored in a database.

The compounds in the test library may be obtained in any suitable manner. For example, the compounds in the test library may be selected from a pre-existing set of compounds. Alternatively or additionally, the compound library may contain compounds that have been created in a synthesis process such as a combinatorial synthesis process. The test library of compounds may be synthesized either by solid or by liquid phase parallel methods known in the art. The combinatorial process can be directed by synthetic feasibility without prior knowledge of the biological target. Additionally, compounds may only exist in a virtual sense (i.e. in an electronic form stored on a hard drive or in memory in a computer), such that the compounds' characteristics can be calculated and/or predicted without the compounds being physically present. Selected candidate (second or third tier) molecules can then undergo actual synthesis and testing.

Illustratively, a new compound data set consisting of 15,000 compounds can be created using, for example, combinatorial synthesis. The new compound data set can be compared to a pre-existing data set stored in a database such as an Oracle™ relational database management system. The relational database management system may store numeric data, alphanumeric data, binary data (such as in e.g., image files), chemical data, biological activity data, analytical models, etc. Members of the new compound data set that are not redundant of the pre-existing compound data set can then be retained and added to the database containing the pre-existing compound data set. The compound data set thus defined forms the testing library.

A commercial software package such as ISIS™ (Integrated Scientific Information System—a commercially available client/server application from MDL™ Information Systems, Inc., San Leandro, Calif.) can be used to compare data sets. ISIS™ can interface with, e.g., an Oracle™ database to allow for the searching of, for example, chemical data and structures stored in the Oracle™ database. ISIS™ allows a user to compare two compound data sets and determine the overlap (redundancy) between the data sets. Moreover, it allows the registration of redundant non-structure related data into the database while retaining only unique structure information. Of course, in other embodiments, data sets of compounds need not be compared to form a test set. For example, a number of compounds can be formed by a combinatorial synthesis process and then may be characterized. The compounds may form a test set without comparing the newly formed compounds with a preexisting compound data set.

After forming the test library, some or all of the members of the compounds in the test library may be evaluated according to a predetermined pharmaceutical or a therapeutic profile. The evaluation can be conducted using, for example, Sybyl™, a commercially available molecular modeling suite of programs from Tripos, Inc., St. Louis, Mo. Using Sybyl™, 2D structural information can be transformed into 3D coordinates, and physicochemical properties based on either 2D or 3D chemical information can be obtained. 2D or 3D information can be used to determine if a compound is to be assigned a particular pharmaceutical or therapeutic profile. Using the pharmaceutical or therapeutic profile, only those compounds that fit the profile may be selected, and compounds that do not fit the profile are excluded, thus reducing the number of potential candidates. The selection of compounds using the pharmaceutical or therapeutic profile can take place before or after the analytical model is formed.

A typical pharmaceutical profile includes characteristics that make a compound desirable as a pharmaceutical agent. For example, one characteristic of a pharmaceutical profile may be the ability of a compound to dissolve in a liquid. If a compound dissolves in such liquid, then the compound fits the pharmaceutical profile. It is does not, then it does not fit the pharmaceutical profile. A typical therapeutic profile includes characteristics that make a compound desirable for a particular therapeutic purposes. For example, if the particular therapeutic purpose is to provide therapy to the brain, then the compound may have characteristics (e.g., small size) that permit it to pass the blood-brain barrier in a person. If the compound has these characteristics, then it fits the therapeutic profile. Characteristics relating to the pharmaceutical or therapeutic profile may be present in the test library and may be stored in a database along with each of the compounds in the test library. At any point, the profile information may be used to select compounds that have a higher likelihood of exhibiting a predetermined biological activity and/or are suitable for the particular pharmaceutical or therapeutic goal in mind.

II. Test Set and Training Set Selection

A test set of compounds and a training set of compounds are selected from the test library of compounds. Typically, the number of compounds in the training set is less than 20% of the number of compounds in the test set. After the training set is formed, the test set may be the remaining compounds in the test library. For example, a test library may contain 700,000 molecules and the formed training set may consist of 15,000 molecules. The test set may then consist of the remaining 685,000 molecules.

The information content of the training set, whether a combinatorial library candidate for HTS or a statistical analysis data set, influences the efficiency and/or utility of the analysis methodology. For this reason different experimental design strategies have been developed for diverse compound selection from a larger chemical library or chemical diversity space. (Hassan, M. et al., Mol. Diversity, 2:64-74 (1996); Higgs, R. E. et al., J. Chem. Inf. Comput. Sci., 37:861-870 (1997).

In some embodiments, a diverse selection (DS) process can be performed using a D-optimal design strategy (Euclidian distance metric, Tanimoto Similarity Coefficient, 10,000 Monte Carlo Steps at 300 K, with a Monte Carlo Seed of 11122, and termination after 1,000 idle steps), as implemented in Cerius²™ (version 4.0; Molecular Simulations Inc., San Diego, Calif.). In a DS process, compounds are selected to maximize representation in the test library. For example, if the compounds have characteristics that make them cluster in some way (e.g., by similar morphology), then fewer compounds in the cluster are selected in order to increase the representation of other compounds in the training set.

In other embodiments, a diverse selection of 5,000 compounds was randomized with regard to the biological activity, yielding a diverse/randomized (DR) training set. The compounds in the diverse/randomized (DR) training set are randomly assigned biological activities, and a model is created. If the created model does not perform well, then the selected training set is desirable since the biological activities were randomly assigned and were not derived from actual testing. For example, 10 independent rounds of randomization can be performed where compounds are randomly (using a random number generator) assigned to the activity bins proportionately to their initial distribution, but without regard to their chemical structure and their measured biological activity.

In other embodiments, a random (RS) selection process can be used to form the training set. A training set formed by a random selection process is a stochastic sampling of a complete library, and therefore represents the information content in proportion to its distribution in the test library. In a sense, the information content is lower in a training set formed by random selection than by diverse selection. In a random selection process, densely populated areas with repetitive information are sampled more frequently than sparsely populated areas containing unique information.

III. Assaying

The compounds in the training set may be assayed to determine their biological activity. In some embodiments, an ion channel assay may constitute a homomultimeric, or heteromultimeric isoform of a single ion channel, or multiple ion channels related through their gene sequence (i.e., a “gene family”). If an assay constituting a homomultimeric or heteromultimeric ion channel of the same gene family is used, it is possible to establish a “gene family library space” by intersecting the screening results for different ion channel types (i.e., intersecting models). A “gene family library space” refers to a library consisting of compounds that work against more than one type of ion channel. For example, compounds in a gene family library space may work against two or more types of ion channels. A “gene specific library space” may be formed by subtracting the results of different screening results for different ion channel types (i.e., differentiating models). A “gene specific library space” refers to a library consisting of compounds that work preferentially against one type of ion channel.

The biological activities determined by the assaying process may be defined by two or more classes (e.g., high activity and low activity). Preferably, the biological activities may be defined by three of more related classes (e.g., high activity, moderate activity, and low activity). For example, the screening assay determines the biological activity of each compound. Each compound is then assigned to a particular class with a predetermined activity range, based on the determined biological activity. In some embodiments, the activity ranges for the different classes may include “high activity”, “moderate activity”, “low activity”, and “inactive”. The skilled artisan can determine the quantitative bounds of the classes.

The present inventors have found, rather surprisingly and unexpectedly, that improved predictability can be obtained by classifying activity data into more than two classes of biological activity. As shown in the Examples below, embodiments of the invention exhibit significantly improved predictability in comparison to, for example, conventional binary recursive partitioning processes. Embodiments of the invention represent an improvement over the methods published by Gao and Bajorath, Mol. Diversity, 4:115-130 (1999) (discussed below).

Any suitable assay known in the art may be used to determine the biological activity of the compounds in the test library. For example, the biological activity of the compounds may be determined using a high-throughput whole cell-based assay.

In preferred embodiments, the assay determines the ability of the compounds in the test set to modulate the activity of ion channels and the degree of activity. For example, the activity of an ion channel can be assessed using a variety of in vitro and in vivo assays, e.g., measuring current, measuring membrane potential, measuring ligand binding, measuring ion flux, e.g., potassium, or rubidium, measuring ion concentration, measuring second messengers and transcription levels, using potassium-dependent yeast growth assays, and using, e.g., voltage-sensitive dyes, ion-concentration sensitive dyes such as potassium sensitive dyes, radioactive tracers, and electrophysiology. In a specific example, changes in ion flux may be assessed by determining changes in polarization (i.e., electrical potential) of the cell or membrane expressing the potassium channel. A preferred means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g. Ackerman et al., New Engl. J. Med. 336:1575-1595 (1997)). Whole cell currents are conveniently determined using the standard methodology (see, e.g., Hamil et al., Pflügers. Archiv. 391:85 (1981).

In an illustrative assay for a potassium channel, samples that are treated with potential potassium channel modulators are compared to control samples without the potential modulators, to examine the extent of modulation. Control samples (untreated with activators or inhibitors) are assigned a relative potassium channel activity value of 100. Modulation is achieved when the potassium channel activity value relative to the control is distinguishable from the control. The degree of activity relative to the control is generally defined in terms of the number of standard deviations from the mean. For instance, if the mean is 0%, and the standard deviation is 25%, then the activity ranges could be defined as 1) 0-25%, i.e. within 1 standard deviation of the mean, 2) 25-50%, i.e. within 2 standard deviations from the mean, 3) 50-75%, i.e. within 3 standard deviations from the mean, and 4) 75-100%, i.e. within 4 standard deviations from the mean. These ranges of activity may correspond to, for example, inactive, weakly active, moderately active, and highly active, respectively.

III. Analytical Model

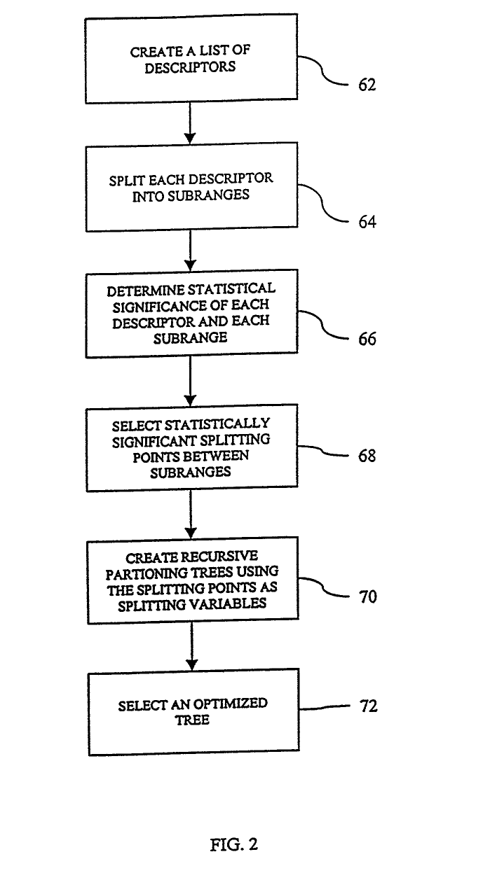

Referring to FIG. 2, a list of descriptors is created to form a descriptor space (step 62). A descriptor may be binary in nature, i.e. it can denote the presence or absence of a feature but not its extent. For example, a descriptor named “heterocyclic” may denote the presence (1) or absence (0) of heteroatoms in a ring otherwise constituted by carbon atoms, but holds no information as to the number of heteroatoms present. Alternatively, a descriptor could be a continuous range descriptor. That is, it can denote the extent to which a particular feature is represented. For example, the molecular weight of a compound may be considered a continuous range descriptor. All molecules have a molecular weight, but the extent of the descriptor (e.g., a molecular weight as expressed in a range of Daltons) can be used to discriminate one molecule from another. Other examples of descriptors include the principal moment of inertia in a molecule's primary X-axis (PMI_X), a partial positive surface area (JURS_PPSA_{—}1), molecular density (Density), molecular flexibility index (phi), etc. In embodiments of the invention, hundreds or thousands of such descriptors can be considered when forming an analytical model.

A number of exemplary descriptors are provided in Cerius²™, commercially available from Molecular Simulations, Inc., San Diego, Calif. Cerius²™ is capable of generating descriptors such as spatial descriptors, structural descriptors, etc. for evaluation. It is also capable of creating recursive partitioning trees. It also allows for the variation of variables such as knot limit, tree depth, and splitting method. In embodiments of the invention, the tree depths of the recursive partitioning trees created are systematically varied until the optimal tree(s) are determined.

Each descriptor is subjected to a process called splitting, in which the range (highest descriptor value minus lowest descriptors value) is split into subranges (step 64). By systematically varying the splitting process, the statistical significance of each descriptor and its correlated range is determined (step 66). Splitting points are identified by systematically evaluating the subranges for the possibility to divide the compounds into statistically differentiated subsets based on their assigned category (step 68). The statistically most significant splitting point then becomes a splitting variable in the recursive partitioning tree.

Illustratively, a descriptor such as molecular weight can be optimized. Based on past experience or knowledge, it may be determined that the molecular weight of the particular modulator being sought would have a molecular weight ranging from 23 to 20,000. The range of 23-20,000 can then be split into progressively smaller subranges. The training set data are then applied to these splits to determine which subrange is the optimal range. For example, if it is discovered that out of 200 candidate compounds, 50 compounds having a molecular weight between 23-10,000 exhibit high activity and 150 compounds having a molecular weight between 10,000 and 20,000 exhibit low activity, then the range of 23-10,000 is selected as the more preferred range. Since a molecular weight of 10,000 splits the data, it is a splitting point and may be referred to as a “knot”. “Splitting points” and “knots” are used interchangeably and refer to values that are used to split a range for a descriptor. The 23-10,000 molecular weight continuous range descriptor is then used as a splitting variable at a node in a classification and regression tree. For example, the variable MW (molecular weight) could be used in two consecutive splits: MW<=10,000 and MW>23, to define the preferred range of 23-10,000 used to classify compounds in the test set. In this example, only one descriptor with two knots is described for simplicity of illustration. However, in other embodiments, the number of knots per descriptor may be 2 to 140 or more. Narrow or broad ranges for the descriptors can be evaluated for statistical significance.

A. Forming Trees

A plurality of recursive partitioning trees is created (step 70). Tens or hundreds of trees may be generated in some embodiments. Each tree uses the descriptors, as calculated and optimized above, as splitting variables to form splits in the trees. Many such trees are created while varying such parameters as the knot limit, tree depth, and splitting method. Then, an optimal tree is selected (step 72) as an analytical model. The most desirable tree found is the one that differentiates the data the best according to biological activity.

In a typical recursive partitioning tree, parent nodes are split into two child nodes. A splitting variable splits the training set compounds into two statistically significant groups, and these two groups are classified into two respective child nodes. A Student's t-test may be used to determine the statistical significance of the split. In forming a tree, splitting methods such as the Gini Impurity, Twoing Rule, or the Greedy Improvement can be used to split the compounds. These methods are well known in the art and need not be described in further detail here (see: Breiman, L., Friedman, J. H., Olshen, R. A., Stone, C. J. Classification and Regression Trees, Wadsworth (1984)).

Once a best split is found, the classification and regression tree process repeats the search process for each child node, continuing recursively until further splitting is impossible or stopped. Splitting is impossible if only one case remains in a particular node or if all the cases in that node are of the same type. Alternatively, the process ends when there are either no more significant splits to be obtained, or when the minimum number of compounds per node is reached. The nodes at the bottom of a tree (i.e., where further splitting stops) are terminal nodes. Once a terminal node is found, the node is classified. The nodes can be classified by, for example, a plurality rule (i.e., the group with the greatest representation determines the class assignment). The tree may be pruned to the appropriate tree depth as defined at the outset of the process.

Sometimes, a molecule is included in a node because one of its descriptors increases the probability for it to be classified as “highly active”. If this molecule, by virtue of its measured activity, belongs to a class other than the one to which it has been assigned, then that molecule is a “false positive” within that node. This can occur with a series of similar (congeneric) compounds. Conversely, molecules may have been eliminated from a node based on dissimilarity, but should have been included. These molecules are “false negatives”. Models try to minimize both the number of false negatives and false positives.

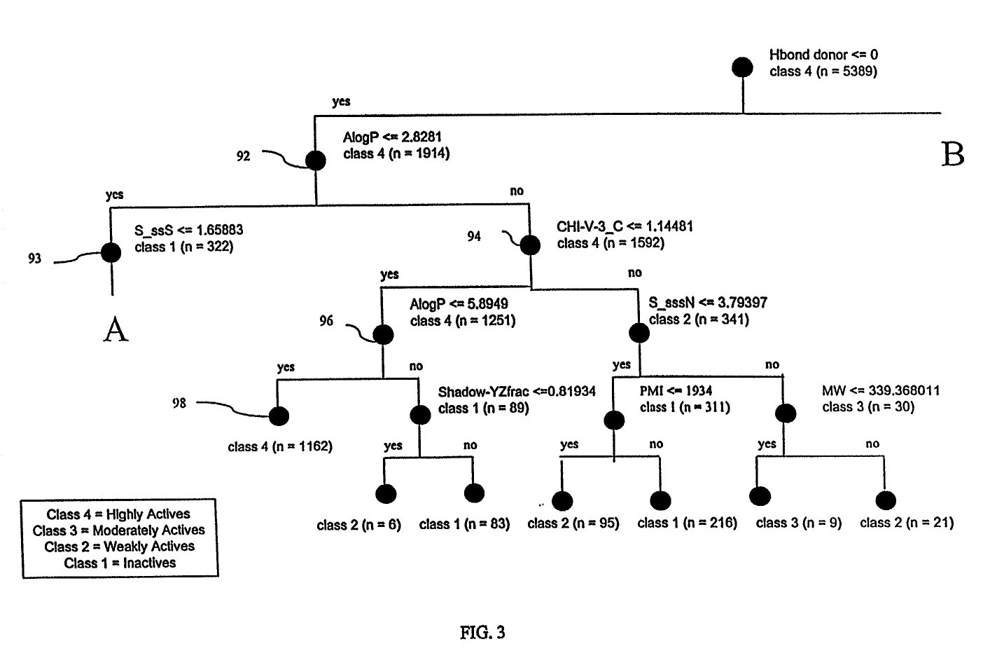

FIG. 3 shows an example of a portion of a recursive partitioning tree. The area where the letters “A” and “B” are present would have additional nodes, branches, etc. For purposes of clarity, these additional tree structures have been omitted. In this example, a node 92 may be characterized as a highly active node where the tree initially classifies 1914 members of a test set as being highly active. Then, the splitting variable “AlogP<=2.8281” may be applied to the 1914 compounds at the node 94. “AlogP” is a property of a chemical compound that is described in greater detail in Ghose A. K. and Crippen G. M. J. Comput. Chem., 7, 1986, 565. Compounds that satisfy this condition are placed in node 93 while compounds that do not are placed in node 94. The compounds assigned to these nodes 93, 94 are further split in a similar fashion, but with different rules. The classification of each node 93, 94 can be determined by determining which particular activity (i.e., highly active, moderately active, weakly active, or inactive) predominates at the node. The compounds can be split until a terminal node 98 is reached. In some embodiments, the terminal node may contain compounds, which all (or a majority of) have the same biological activity. The terminal node may then be characterized by the determined biological activity. In this particular example, the nodes 92, 94, 96, 98 are all characterized as highly active nodes. The compounds classified in the terminal node 98 satisfy the following conditions:

| | |
|---|---|
| Hbond donor <= 0, yes | (“Hbond donor” is the number |
| | of hydrogen bond donors) |
| AlogP <= 2.8281, no | (“AlogP” is a calculated octanol/water |
| | partitioning coefficient) |
| CHI-V-3_C <= 1.14481, yes | (“CHI-V-3_C” is a 3rd Order Cluster |
| | Vertex Subgraph Count Index) |
| AlogP <= 5.8949, yes | (“AlogP” is a calculated octanol/water |
| | partitioning coefficient) |

This set of rules or descriptors can be used to select a class of compounds that are expected to have a “high biological activity”. In this example, the 1162 compounds in the terminal node 98 may serve as potential candidates for modulators. If desired, these compounds may be analyzed (e.g., by a computer or the skilled artisan) to determine if there are any chemotypes that are prevalent in the terminal node compounds. These chemotypes may serve as a basis for further research or analysis. Advantageously, in embodiments of the invention, potentially effective chemotypes can be identified in addition to providing enhanced hit rates.

B. Selecting an Optimal Tree

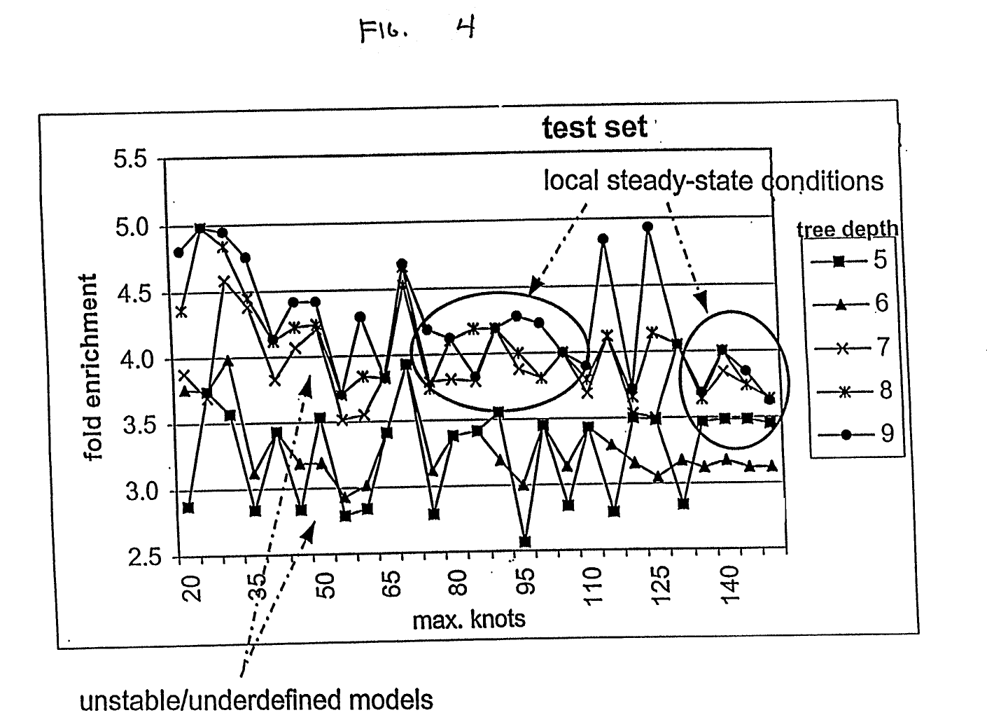

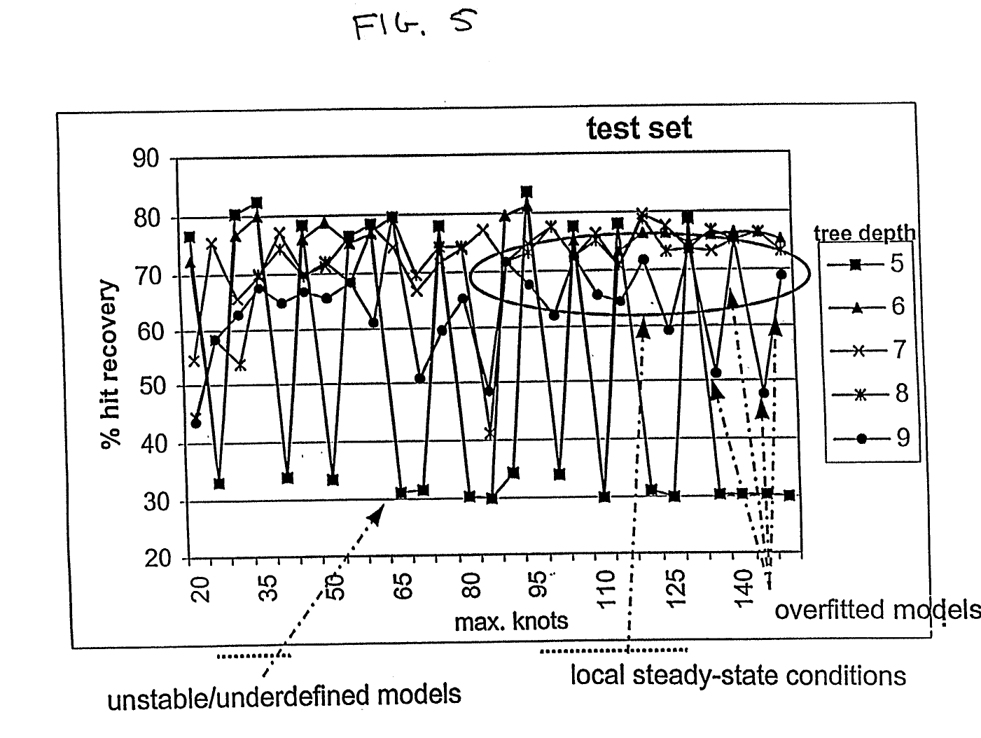

There are two distinct measures for determining the success of a recursive partitioning analysis. The following parameters can be used to determine the optimal tree: “fold enrichment” refers to the % correctly predicted “hits” divided by the % empirically determined “hits”, where the definition of “hit rate” is dependent on the class assignment (vide supra). A “hit” might be, for example, a classification in a class characterized by “highly active”. Exemplary optimization traces for fold-enrichment are represented in FIG. 4. “% class correct” for the training set and the corresponding “% hit recovery” for the test set refer to the number of compounds correctly predicted to be “highly active” as a percentage of the total number of compounds known to be “highly active” (in the training set and test set, respectively). Exemplary optimization traces for % hit recovery are represented in FIG. 5. “retrieval rate” refers to the number of compounds classified by the recursive partitioning model as having an increased probability of being “highly active”, expressed as a percentage of the total number of compounds under consideration in the test set.

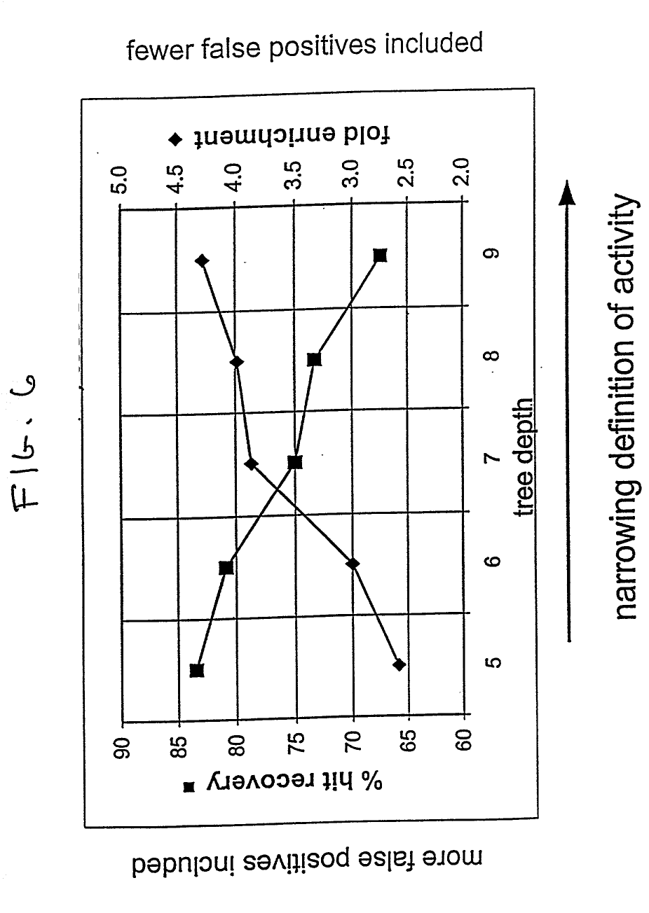

As shown in FIG. 6, fold enrichment and % hit recovery are not necessarily independent, rather they are interdependent. As the models become more sophisticated, e.g., increased tree depth, the activity is more narrowly defined, and as a result more false positives (compounds initially incorrectly included as active, but by a more refined model correctly identified as inactive) are eliminated from the model. However, concurrently, the method also eliminates more false negatives (compounds initially correctly identified as active, but subsequently incorrectly classified by the model as inactive), resulting in a better fold enrichment in the remaining models, but a lower overall % hit recovery.

In order to evaluate whether a method exhibits stable behavior or yields variable models due to perturbations, a reference frame was established. One commonly employed reference frame is the running average of a centroid and its adjacent neighbors. The following example can be used to illustrate how to calculate running averages and how to determine centroids. Given a series 1, 2, 6, 16, 18, 20, 22, 23, 24, 22, the running averages with a window size of 1 would be: 3, 8, 13.3, 18, 20, 21.7, 23, 23; the centroids would be 2, 6, 16, 18, 20, 22, 23, 24. The (first order) derived value “absolute value of the differential (running average minus centroid)” then reflects the local variability of the function. If this value is close to zero, it indicates a “local steady-state”. For example, with respect to the above-described example, the absolute value of the differential(running average minus centroid) would be 1 (3−2), 2 (8−6), 2.7 (13.3−16), 0 (18−18), 0 (20−20), 0.3 (21.7−22), 0 (23−23), and 1 (23−24). It is not intuitive that the series stabilizes towards the end of the series. The present inventors have determined that this “steady state” can be used as a basis for selecting an optimized model. In another more specific example, three consecutive models (e.g., viewed consecutively along the x-axis of a graph) may have fold enrichments of 4.1, 4.2, and 4.3, respectively. The centroid may be the central point 4.2 and the running average of these three points may also be 4.2. The absolute value of the differential for these values is 0 (i.e., the absolute value of (4.2−4.2)=0).

To differentiate between a “local steady-state” on an incline (all consecutive increment values are positive) or decline (all consecutive increment values are negative) of the optimization trace and one on a level region of the trajectory (consecutive increment value average out to zero), it is desirable to evaluate the “first derivative vs. the knot limit” of the (second order) function. (See, e.g., Elementary Differential Equations and Boundary Value Problems, Boyce, W. E., DiPrima, R. C., Wiley & Sons, NY, 1969.)

However, it is rather straightforward to inspect a curve visually and distinguish between the three possibilities. If both the fold enrichment and the % class correct converge on the “local steady-state” defined by a particular knot limit, then that knot limit is presumed to be the minimally acceptable knot limit. A “local steady-state” can be evaluated on three consecutive differential values, i.e. a knots span of 5 consecutive steps. This is equivalent to 3 consecutive running averages, and spans a total of 20 knots between the highest and the lowest conditions in the series.

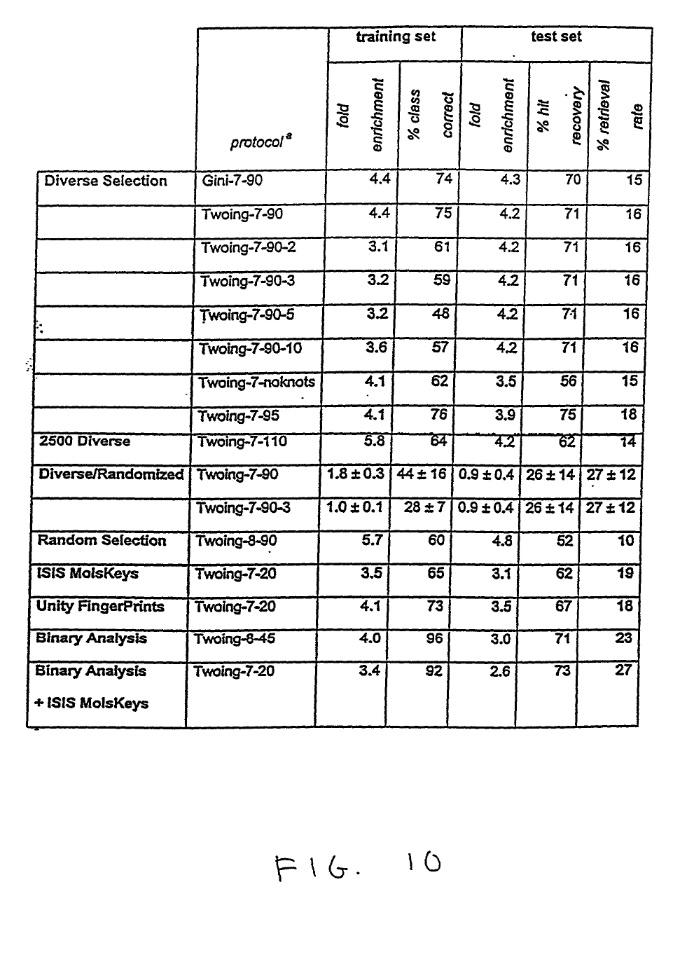

The inventors have found empirically, that by defining a “local steady-state” (FIGS. 4 and 5) as variations of less than about 0.1-fold enrichment and less than about 7% class correct (preferably less than about 2%), many of the areas with irregularities could be eliminated. Recursive partitioning models selected with these criteria also tended to be more predictive for the test set, in both fold enrichment and % hit recovery. These values are slightly more restrictive than, but in general agreement with, a standard deviation of 0.1-fold and 7% obtained during the randomization and cross-validation experiments (FIG. 10:Twoing 7-90-3). In short, the intermodel variation is preferably less than about 0.1-fold enrichment and less than about 7% class correct (preferably less than 2%). In some embodiments, the chosen model is the first running average, or the third original model to satisfy these criteria (i.e. it is the one in the center of the running average of 5 consecutive runs). However, in other embodiments, any of the models that are used to define the local steady-state may be used as a model as each of the models used to define the steady-state are generally stable models.

IV. Selecting Compounds Using Profiles and Assays

In embodiments of the invention, a subset of compounds is selected using the analytical model and a pharmaceutical or therapeutic profile. As noted above, compounds in the test library may be assigned characteristics of a particular pharmaceutical or therapeutic profile. After the analytical model is formed and is applied to the test set of compounds, the desired pharmaceutical or therapeutic profile is applied to the first subset of compounds to form a second set of compounds. This excludes compounds that do not satisfy the desired pharmaceutical or therapeutic profile.

The subset of compounds may then be screened in a second assay to verify the biological activity of the first or the second subset of compounds. The second assay may be the same or a different assay that is performed on the training set (described above). By performing a second assay on the second subset of compounds, a third subset of compounds may be formed. The above-described analytical model determines which compounds in the test set have a high likelihood of having biological activity. The second assay is used to verify the biological activity of the compounds in the second subset of compounds. In some embodiments, only those compounds exhibiting the desired biological activity are selected for inclusion in the third subset. The compounds in the third subset may be investigated for use as potential modulators for ion channels.

The third subset may constitute the set of compounds that exhibit high biological activity. Compounds may be selected and then stored in an appropriate database to form one or more specific libraries that may be stored in one or more databases. For example, the libraries may be gene-family libraries or gene-specific libraries that are stored in one or more databases. These libraries are described above. The compounds may then be extracted from these specific libraries and further tested as, for example, possible drug candidates.

Functions such as the selection of compounds using a therapeutic or pharmaceutical profile, the creation of the analytical model (i.e., the creation of descriptors or trees, and the optimization and/or selection of models), the application of the analytical model to a test set, etc. can be performed using a digital computer that executes code embodying these and other functions. The code may be stored on any suitable computer readable media. Examples of computer readable media include magnetic, electronic, or optical disks, tapes, sticks, chips, etc. The code may also be written in any suitable computer programming language including, C, C++, etc. The digital computer used in embodiments of the invention may be a micro, mini or large frame computer using any standard or specialized operating system such as a UNIX, or Windows™ based operating system. Moreover, any suitable computer database may be used to store any data relating to the test library, test set, training set, or analytical models. Preferably, a computer database such as an Oracle™ relational database management system is used to store this information.

### EXAMPLES

Analytical models were created and applied to a test set of compounds. In the examples described below, a pharmaceutical or therapeutic profile was not applied to the compounds selected by the analytical models. 20,986 compounds were selected from a chemical library selected for screening. The 20,986 compounds formed a test library. The chemical library was composed of combinatorial chemistry derived compounds, synthesized either by solid or by liquid phase parallel methods. The biological activity of all test library compounds was determined individually.

A set of eight randomly selected plates, accounting for 640 library members, was analyzed by LC/MS (liquid chromatography/mass spectrometry). Of the total number of samples analyzed, 81% were found to be better than 80% pure, and 66% were found to be better than 90% pure. The median, average, and standard deviation values were 94%, 88%, and 15%, respectively. Therefore, the purity of the majority of the library members was deemed to exceed 80%.

The combinatorial process was directed by synthetic feasibility without prior knowledge of the biological target. Since the chemical library was set up to take advantage of synthetic feasibility rather than molecular diversity, a diversity analysis prior to compound selection was not performed.

The compounds in the test library were divided into a 5,000 member training set based on either Diverse Selection (DS; D-optimal Design strategy), and a 15,985-member test set. Biological data were generated in a high throughput screening (HTS) process using a cell-based method.

After screening, the training set members were subsequently assigned to activity bins based on their relative biological activity. For the quaternary analysis, they were assigned as follows: 147 in class 4 (“highly active”), 471 in class 3 (“moderately active”), 912 in class 2 (“weakly active”), and 3,470 in class 1 (“inactive”). For the binary analysis, they were assigned as follows: 147 in class 4 (“active”), and 4,853 in class 1 (“inactive”). No attempt was made to identify false positives or negatives.

The Diverse Selection of 5,000 compounds (DS) was randomized with regard to the biological activity, yielding the Diverse/Randomized (DR) training set. To that purpose 10 independent rounds of randomization, were performed where compounds were randomly (using a random number generator) assigned to the activity bins proportionately to their initial distribution, but without regard to their chemical structure and their measured biological activity.

1,387 descriptors were generated for each of the 20,986 members of the chemical library. Initially, 229 descriptors, distributed over the following categories, were calculated using the commercially available implementation of Cerius²™ (version 4.0; Molecular Simulations Inc., San Diego, Calif.): fragment constants, conformational descriptors, electronic descriptors, graph-theoretic descriptors, topological descriptors, information-content descriptors, spatial descriptors, structural descriptors, and thermodynamic descriptors. Then, 166 public ISIS™ MolsKeys were generated using ISIS™/Host (version 3.0; MDL™ Information Systems Inc., San Leandro, Calif.), and 992 2D FingerPrints were generated using Unity™ (version 4.0; Tripos Inc., St. Louis, Mo.).

The RP conditions were varied systematically using Cerius²™. The defaults as implemented in Cerius²™ are in boldface. The following conditions were considered: Weighting by Classes (not varied), i.e. each class is considered of equal importance to the model rather than each compound, Splitting Method: Twoing/Gini/Greedy, i.e. the formalism that determines how groups are divided or partitioned into statistically distinct nodes or subgroups; maximum tree depth=5/6/7/8/9/12/16/20, i.e. the maximum number of splits that may occur before the partitioning process terminates; Pruning: Moderate (not varied), i.e. the procedure that determines the appropriate statistically significant tree depth for each node; minimum number of samples per node=1/110^{th} (not varied), i.e. a node or subgroup cannot contain fewer than 1% of all compounds in the training set; maximum number of knots per split=20−150 (in increments of 5), i.e. the maximum number of ways a descriptor range may be divided before statistical relevance is determined; and when applicable: number of cross-validation groups=2,3,5,10, i.e. the number of groups used to test the statistical stability or significance of the model conditions. Therefore, any particular set of conditions can be characterized by splitting method—maximum tree depth—maximum number of knots —number of cross-validation groups (when applicable).

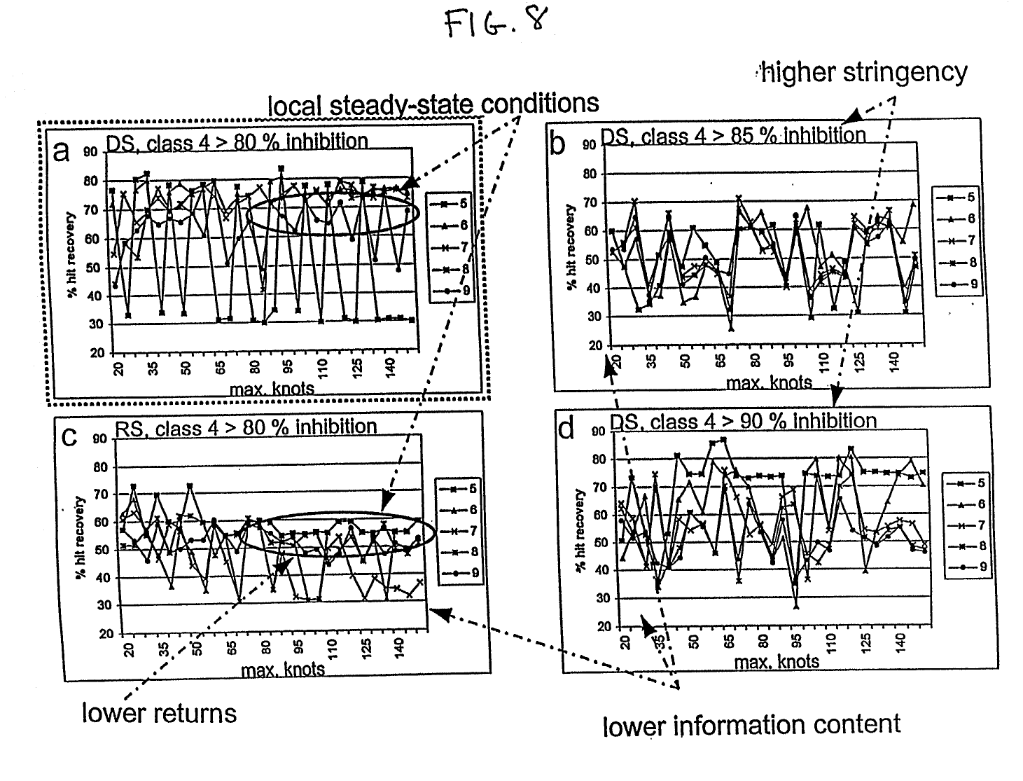

In one experiment, the RS training set (Twoing-8-90; the “8” refers to tree depth and “90” refers to the maximal knot limit) predicted 5.7-fold enrichment, 60% class correct, and yielded 4.8-fold enrichment, 52% hit recovery (FIG. 10). The RS training set was even less predictive, and unstable behavior at a tree depth of either 6 or 7 was found (FIG. 8c). Although the fold enrichment, which reflects the density of the information matrix, compares favorably with the DS training set (4.2-fold when taken at Twoing-7-90, see FIG. 10) both the % hit recovery and % retrieval rate, which reflect the information content are decreased. This probably is a reflection of the elimination of tentative false positives from the prioritization list.

The efficiency of the RP process can be expressed either as fold enrichment, or as % class correct or % hits retrieved for the training set and the test set, respectively. Ideally, the numbers for the training set and the test set match closely, i.e., the model shows good overall predictivity.

One method for determining the success of a model, Consensus Scoring, emphasizes increases in hit rate by eliminating false positives from the prioritization list. (Charifson, P. S. et al., J. Med. Chem., 42:5100-5109 (1999)) However, this only addresses enhanced hit rates, and does not address, or only narrowly addresses, the following goals: 1. to increase the efficiency of primary screens, i.e. increased hit rates; 2. to identify and pursue multiple chemotypes in order to develop compounds along parallel product lines, i.e. to achieve the highest % chemotypes retrieved possible; and 3. to explain nonlinear structure-activity relationships. Other factors such as the cost of a compound collection (Young, S. S. et al., J. Chem. Inf. Comput. Sci., 37:892-899 (1997)) may also contribute to the overall efficiency of the method, but are not explicitly considered in this analysis.

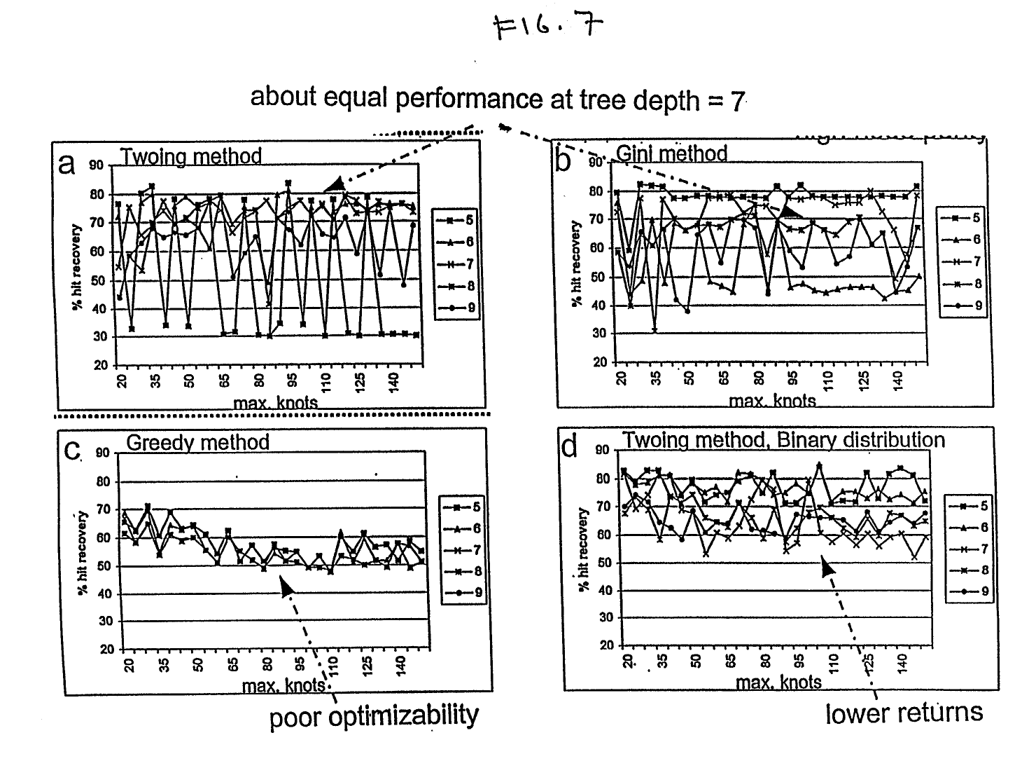

Furthermore, the variability and reliability within the protocol was considered. In general, a low knot limit and small tree depth contribute to unstable behavior, whereas a high knot limit and large tree depth contribute to overfitting and add to computational expense (FIGS. 4 and 5). Conditions that reliably and reproducibly yielded a model at an acceptable computational cost were identified. The Twoing method (FIG. 8(a)) balances the distribution of the branches of the tree, whereas the Gini method (FIG. 7(b)) strives for the highest node purity. (Breiman, L. et al., Classification and Regression Trees, Wadsworth (1984)) When these two methods converge with regard to the tree depth, it can be argued that a suitable tree depth has been obtained. In the DS model, the maximal tree depth at which this occurs is 7 (FIG. 5). Conversely, the Greedy method shows poor optimizability, and a low tree depth and knot limit results in a less predictive model (FIG. 7(c)).

The minimal knot limit in the DS optimization protocol at a maximal tree depth of 7 was determined to be 90. The resulting values (Twoing-7-90) are 4.4-fold enrichment and 75% class correct for the training set, and 4.2-fold enrichment, a 71% hit recovery, and a 16% retrieval rate for the test set (FIG. 10). Since the data obtained from the test set are a relatively close reflection of the data from the training set, it is very likely that this approach is suited to select valid and predictive methods. Because the optimal conditions are inherently dependent on the training set, changes in the training set, such as changes in the chemical composition, or in the biological data, such as a different target selection, may require reoptimization of the RP conditions.

In addition, the built-in autoselection protocol in Cerius²™, i.e. the “no knot limit” setting was examined. It yielded the following data: Twoing-7-noknots predicted 4.1-fold enrichment, 62% class correct, and yielded 3.5-fold enrichment, 56% hit recovery, and a 15% retrieval rate (FIG. 10). The discrepancy between optimal conditions and those selected by the program probably find its roots in the undisclosed optimization criteria of this particular implementation. Unexpectedly, the Twoing-7-noknots protocol has a lower predictive capability for this data set than the models with manually and empirically determined optimal conditions.

When evaluating the efficiency of any methodology, one has to take into account how predictive the method developed on the training set is, when applied to the test set. In parametric methods, this usually is quantitated in the form of correlation coefficients and cross-validation values. Due to the noncorrelative nature of nonparametric methods in general, regression has so far been impracticable. The validation method implemented in Cerius²™ is cross-validation. In addition to the cross-validation experiments described below, 10 independent randomization trials were performed to remove selection bias.

The results of the randomization experiment (n=10; Twoing-7-90) are presented in FIG. 10. Under these conditions, recursive partitioning apparently overstates its efficiency with regard to a fully randomized training set. This may be a result of the distribution of chemotypes in the training and test sets. More importantly, cross-validation of the DR training set (Twoing-7-90-3; “3” refers to the number of cross-validation groups used) yielded results that are in good agreement with results obtained with the test set. This further supports the notion that there is a bias present in the training set that is not present in the test set. The only difference in composition between the test set and the training set is a mathematically introduced one. The mathematical process that introduced this bias must have been the Diverse Selection process that separated the training set from the test set.

The cross-validation experiment led us to investigate how the “information content” of the training set influences the outcome of the analysis. It was found that at a low number of cross-validation groups (2 or 3), i.e. high information dilution, the predictivity of the models fell short of the expectations based on a larger number of cross-validation groups (5 or 10). When the cross-validation experiment was run with 5 cross-validation groups, i.e. 80% of the training set, the model values of the training set and the test set were in good agreement (FIG. 10). Alternatively, when 2 cross-validation groups were used, i.e. 50% of the training set, the cross-validation model was less predictive of the full model. A similar effect is seen when the full training set is reduced to 2,500 diversely selected compounds from the original 5,000 member diverse selection training set. Twoing-7-110 predicted 5.8-fold enrichment, and yielded 4.2-fold enrichment (FIG. 10), but with rather unstable optimization traces (not shown), and a significant discrepancy between predicted and realized yields. This less predictive model reflects the loss of information content in the training set selection, and deserves a closer examination.

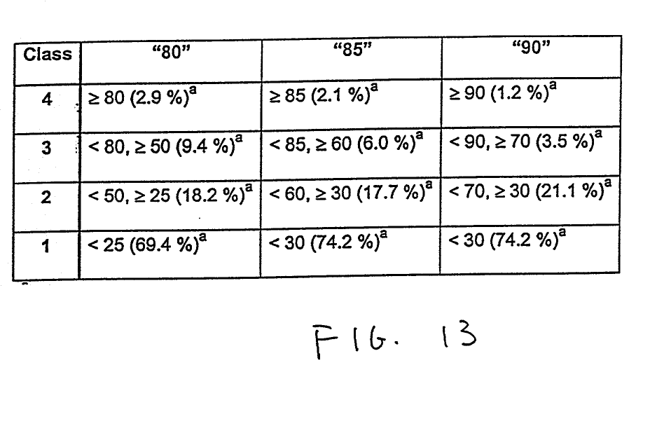

This last point is also illustrated with the results presented in FIGS. 8(a)-8(d). Here, the binning scheme was changed to be more restrictive when assigning compounds to the more active classes (FIG. 13). In FIG. 13, “80”, “85”, and “90” refer to different quantitative thresholds for class 4 (highly active). This had two intended effects: 1. it decreased the initial hit rate, thereby allowing us to focus on the more potent hits only; and 2. it diluted the information content available to the RP algorithm. Surprisingly, the models based on these more restrictive binning schemes were less stable, less predictive, and had an overall lower yield (FIGS. 8(b) and 8(d)). However, these results are entirely consistent with findings from a binary analysis, as discussed below. It probably finds its origin in the fact that prediction accuracy is significantly compromised near any artificial threshold, such as represented by the binning schemes in FIG. 13.

When the criteria and considerations detailed above were applied to the 5,000-member DS training set, the models converged on a tree depth of 7 (FIGS. 4, 5, and 7(a)). The resulting preferred recursive partitioning model (Twoing-7-95) yielded a 3.9-fold enrichment and a 75% class correct for the test set (FIG. 10). The model predicted that 18% of the compounds in the test set belong to the “highly active” class, i.e. a reduction in the number of compounds to be screened by 82%. This 18% retrieval rate should recover more than 18% of the “highly actives” present in the test set (if hits were proportionally distributed between the 18% selected and the 82% remaining compounds), in order to be deemed successful. Indeed, surprisingly and unexpectedly, in using this model, 75% of all “highly active” compounds present were retrieved, thereby enhancing the hit rate some 4-fold. This result satisfies one of the criteria laid out in the introduction: the ability to increase the efficiency of primary screens.

Although 75% of all hits may be retrieved in some cases, all relevant chemotypes may be identified in the process. The identification of chemotypes can lead to the discovery of highly active compounds that may or may not be members of the test or training sets. For example, two or more chemotypes (e.g. heterocyclic molecules having nitrogens, molecules with double bonds, etc.) may be prevalent in a class that is characterized by high biological activity. The identification of chemotypes may be made by one skilled in the art or by a computational apparatus. Compounds of the selected chemotypes that are not in the test set or the training set can be evaluated. In a typical drug discovery process, it is desirable to identify multiple chemotypes. For example, researchers can evaluate compounds of one chemotype. If that chemotype is not particularly effective, then compounds of other chemotypes can be evaluated. Such evaluations can occur in series or parallel. Identifying multiple biologically active chemotypes increases the chances that compounds exhibiting a combination of high biological activity and drug-like (i.e. pharmaceutical) properties will be discovered.

In embodiments of the invention, the distribution of chemotypes within the compound collection may play a role in the performance of the recursive partitioning models. This can impact the desire to pursue multiple chemotypes at the same time in order to develop compounds along parallel product lines. Sometimes compounds can be used for different indications, such as gastrointestinal versus central nervous system diseases. At other times, it can be quite useful to have one lead compound progressing towards the clinic while another one serves as a so-called “back-up” compound. After all, xenobiotics are frequently not readily absorbed, and can be extensively metabolized and excreted.

In a recursive partitioning model, each terminal node represents a different stratification of the data that is not necessarily analogous to, or even consistent with, another node. This opens up the possibility that different nodes may either represent differences in chemical or in biological stratification. The results for each of the terminal nodes were individually investigated. Based on a general definition of chemical core structures, derived from the combinatorial synthetic process, 8 distinct chemotypes could be identified within the training and test sets (CT1 through CT8).

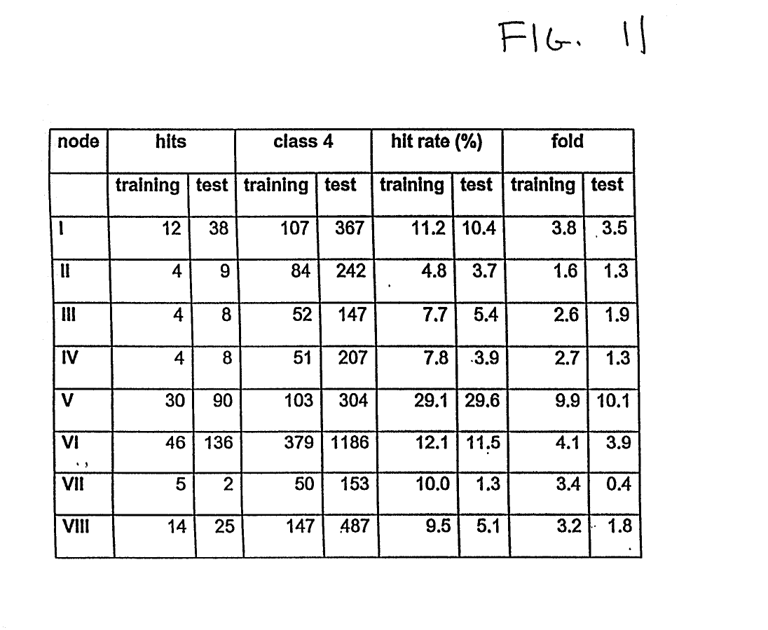

In FIG. 11, data were collected for the terminal nodes in the DS/Twoing-7-90 RP model. It is apparent that there is “significant” variability between the nodes. This may indicate the presence of distinct “binding modes”, or allosterism in the data set. Whereas some nodes, e.g., node V, show robust (about 10-fold) increases infold enrichment in both the training and the test set, other nodes, e.g., nodes II and III, do not perform as well. Moreover, the results for node VII completely miss the mark, which may merely be a reflection of the small number of hits in the training set (5) and the test set (2). In contrast, the results obtained for node VI reflect the overall results, because of the large number of compounds (1186) assigned to that node.

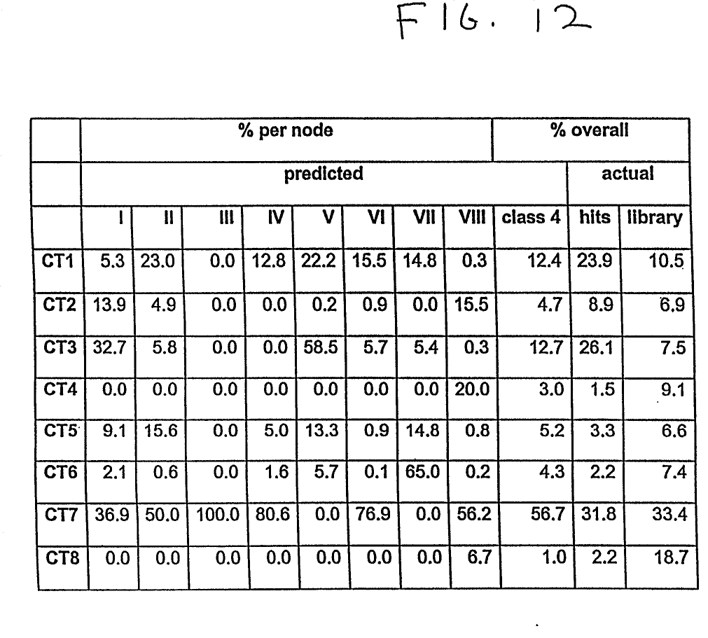

Differentiation by chemotype of the terminal nodes (FIG. 12) indicates that all chemotypes representing “hits” are correctly identified by this method. Although only about 70% of all “hits” are retrieved using this RP model, a full complement of chemotypes was identified. This then leads one to believe that the second goal, which is the option to pursue multiple chemotypes with demonstrated activity against a single biological target, was established. It must keep in mind that the set of compounds identified by the HTS assay as “highly active” may contain some false positives, and equally well may have failed to identify false negatives. The statistical methods could have left out false positives (by chance or by failure to conform to the model), and may have included some of the false negatives. This would be reflected in an overall lower % hit recovery and a higher % retrieval rate.

There seems to be a preponderance of a particular chemotype (CT7) in nodes II, III, IV, VI and VIII, well above the prevalence in the overall distribution. Moreover, this chemotype is lacking in nodes V (which comprises mainly CT1 and CT3) and VII (in majority CT6), whereas CT4 is only present in node VIII. These results support the notion that at least 3, if not more, different “binding modes” may be represented and identified by RP analysis of this data set.

Cross correlation of terminal node and chemotype contributions demonstrates that node V which lacks CT7, but contains a majority of CT3 yields the highest fold enrichment: 10-fold. Conversely, node III consisting of 100% CT7 yields a below average result: 1.9-fold enrichment. These results are indicative of a nonlinear structure-activity relationship within this data set.

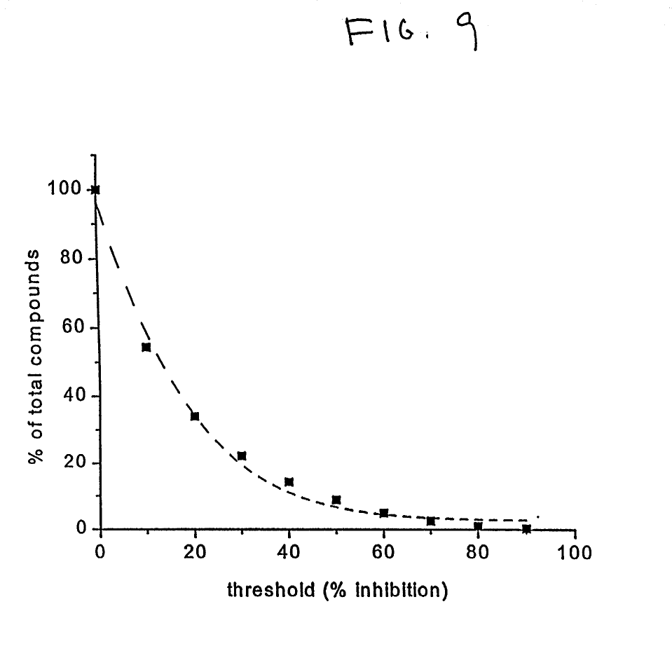

This brings us back to the earlier supposition that the HTS data may not be normally distributed. As can be seen in FIG. 9, the HIS data (plotted points) do not follow a strictly Gaussian behavior (fitted line). Rather, the HTS data have a higher then normal incidence in the 30^{th}-50^{th} percentile range, and a lower than normal incidence at the higher than 70^{th} percentile range. Nevertheless, the central tenet of the central limit theorem is that data sets will appear to be normally distributed as long as the sample size is large enough. At a sample size of over 20,000 data points the data set certainly has a simile of being normally distributed. It does, however, raise the question of whether a collection of multimodal or multiple binding site models could be hidden within this distribution.

Computational chemical methods have focused mainly on describing chemical (diversity) space in 2 dimensions e.g., MDL MolsKeys (McGregor, M. J. et al., J. Chem. Inf. Comput. Sci., 37:443-448 (1997), and 2D FingerPrints (Matter, H. et al., J. Chem. Inf. Comput. Sci., 39:1211-1225 (1999)) as a determinant of biological activity as the chemical composition of a compound to facilitate throughput and ease of calculation (no geometry optimization, and conformer analysis are required for 2D descriptors). Recently, progress has been made to describe compounds in terms of their 3D information content, such as pharmacophore definition triplets (Matter, H. et al., J. Chem. Inf. Comput. Sci., 39:1211-1225 (1999)), or a combination of 2D and 3D descriptors such as those implemented in CODESSA (Menziani, M. C. et al., Bioorg. Med. Chem., 7:2437-2451 (1999)) (Comprehensive Descriptors for Structural and Statistical Analysis). Electrotopological descriptors, as represented by the E-state keys of Kier and Hall (Kier, L. B. et al, Molecular Structure Description: The Electrotopological State, Academic Press (1999)) and implemented in Cerius²™, try to incorporate 2D as well as 3D information by describing the chemical connectivity (topology) of a molecule. Recently, Dixon and Villar (Dixon, S. L. et al., J. Comput.-Aided Mol. Design, 13:533-545 (1999)) reported on their efforts to distinguish 3 different pharmacological classes from those present in the CMC (Current Medicinal Chemistry, MDL Information Systems Inc., San Leandro, Calif.), primarily based on similarity measures. Since they demonstrated superiority of the ISIS MolsKeys over Molconn-X descriptors, the impact of descriptor set selection on the outcome of the RP analysis was investigated.

An experiment was designed where the 166 public ISIS MolsKeys were represented in binary form (0 defines the absence, and 1 the presence of a particular feature), and found that RP (FIGS. 8(a) and 8(b); Twoing-7-20) predicted a 3.5-fold enrichment, and 65% class correct, and yielded a 3.1-fold enrichment, with a 62% hit recovery, and a 19% retrieval rate (FIG. 10). It was not possible to achieve better results with the ISIS MolsKeys than with the original descriptor set. This probably reflects the presence of descriptors in the descriptor set other than the substructurally defined ISIS MolsKeys.

In addition, an experiment based on 992 bitkeys derived from the Unity 2D FingerPrints (2DFP) was designed. Under these conditions (FIGS. 8(a) and 8(b); Twoing-7-20), the algorithm predicted a 4.1-fold enrichment, and 73% class correct, and yielded a 3.5-fold enrichment, with a 67% hit recovery, and a 18% retrieval rate (FIG. 10). Likewise, this probably reflects the absence of physicochemical (whole molecule) and 3D descriptors required for optimal performance by this data set.

Gao and Bajorath (Gao, H. et al., Mol. Diversity, 4:115-130 (1999)) reported that an increase in accuracy from 84% for 2D QSAR to 94% could be obtained using binary QSAR. It was found that RP (Twoing-8-45; FIG. 7(d)) based on a binary distribution decreased both the accuracy (from 75 to 71% hit recovery), and the efficiency (from 3.9 to 3.0-fold) of the models. This reflects a decrease in predictivity of the model rather than an improvement of the training set model, and also results in unstable optimization traces. It is possible that the “fuzzy assignment” approach that was employed, i.e. 4 activity classes rather than just 2, allows the algorithm to compensate for false positive and false negative assignments, without compromising the node purity. A strictly binary classification forces the algorithm to apply penalties to, e.g., compounds having data that fall within a class 3 classification, but which the model assigned to class 4 (the distinction in HTS data between “highly active” and “moderately active” is not necessarily that clear (FIG. 13)). This hypothesis is further supported by the finding of Gao and Bajorath that the prediction accuracy was significantly compromised (about 60% accuracy) near the binary threshold (Gao, H. et al., Mol. Diversity, 4:115-130 (1999)). This problem is exacerbated in the case of percent inhibition data, such as associated with the HTS data set, where the threshold is usually set at the edge of the upper confidence interval, resulting in overlap of the “active” and “inactive” categories whereby most “active” compounds (within the uncertainty) could equally well be placed in the “inactive” category, but only a limited number of “inactive” compounds qualify to be placed in the “active” category. The problem is also compounded by the ceiling effect imposed on the data set by setting a 100% limit as the maximal response, which restricts all compounds passing the threshold to a class 4 assignment, thereby potentially bringing them within the confidence interval of class 3 in a quaternary classification or the “inactive” class of a binary classification.

It was determined if a binary descriptor set would be more appropriate for a binary stratification of biological data by applying the binary classification to the 166 public ISIS MolsKeys. It was found that this did not appreciably improve the predictivity of the models (FIG. 10; Twoing-7-20), nor affect the quality of the models (FIGS. 8(a) and 8(b)).

In summary, embodiments of the invention can also be effectively employed to differentiate between active and inactive compounds in, for example, a test set comprising 20,000, 700,000, or even 1,000,000 compounds (or more), based on data from an experimental HTS assay. Moreover, some embodiments of the invention demonstrate an improved hit rate of the primary screens by about 4-fold, and in doing so correctly identify 75% of all hits, while reducing the size of the chemical library to be screened by over 80%. Furthermore, even though up to 25% of all individual hits go undetected when this particular analysis is employed as a prescreening method, all chemotypes with known activity were correctly identified. This then, opens up the possibility to pursue missed hits and potentially identify false negatives during subsequent screening or SAR (structure activity relationship) development. Other embodiments of the invention demonstrate improved hit rate in the primary screens in excess of 10 to 30 fold.

All publications cited above are incorporated by references for all purposes. None of the publications are admitted to be prior art with respect to the embodiments of the invention.

Moreover, although ion channel modulators are discussed in detail, it is understood that embodiments of the invention are not limited to ion channel modulators. For example, embodiments of the invention can be useful for screening compounds that interact with cell membrane receptors, enzymes, nuclear receptors, as well as for screening compounds that act against pathogens such as bacteria, molds, fungi, and viruses.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention.

What is claimed is:

1. A method for screening compounds for biological activity comprising:
a) selecting a test set of compounds;
b) selecting a training set of compounds;
c) entering training set data into a digital computer, wherein the training set data are derived from a high throughput screening assay on the training set of compounds;
d) forming an analytical model using a recursive partitioning process and the training set data;
e) selecting a first subset of compounds using the analytical model; and
f) selecting a second subset of compounds using a predetermined pharmaceutical or therapeutic profile.

2. The method of claim 1 wherein forming the analytical model comprises:
g) creating a list of descriptors;
h) creating a plurality of trees using the training set data and the descriptors;
i) optimizing the plurality of trees;
j) selecting an optimized tree; and
k) using the optimized tree to select the first subset of compounds.

3. The method of claim 2 wherein creating the plurality of trees comprises, for each tree:
l) identifying a plurality of descriptors;
m) identifying a plurality of splitting points for each descriptor, each splitting point splitting the descriptor into subranges;
n) selecting one of the splitting points for each descriptor that defines a subrange that discriminates the compounds in the training set in a statistically significant manner; and
o) creating a recursive partitioning tree with the splitting variables that are formed using the selected splitting points.

4. The method of claim 1 wherein the high throughput screening assay is for ion channel modulators.

5. The method of claim 1 further comprising:
g) performing a screening assay on the second subset of compounds to form a third subset of compounds.

6. The method of claim 1 wherein f) is performed before e).

7. A method for screening compounds for biological activity using a digital computer, the method comprising:
a) selecting a test set of compounds;
b) selecting a training set of compounds;
c) entering training set data into a digital computer, wherein the training set data are derived from a high throughput screening assay for ion channel modulators on the training set of compounds;
d) forming an analytical model using the training set data and a recursive partitioning process; and
e) identifying a subset of compounds using the analytical model.

8. The method of claim 7 wherein the ion channel modulators are allosteric modulators.

9. The method of claim 7 wherein at least some of the compounds in the test set are formed using a combinatorial synthesis process.

10. The method of claim 7 wherein d) forming the analytical model comprises:
f) identifying a plurality of descriptors;
g) identifying a plurality of splitting points for each descriptor, each splitting point splitting the descriptor into subranges;
h) selecting one of the splitting points for each descriptor, wherein the selected splitting point defines a subrange that discriminates the compounds in the training set in a statistically significant manner; and
i) creating a recursive partitioning tree with the splitting variables that are formed using the selected splitting points.

11. The method of claim 10 further comprising:
j) creating a plurality of recursive partitioning trees;
k) identifying trees defining a predetermined local steady state condition; and
l) selecting one or more of the recursive partitioning trees within the identified trees.

12. The method of claim 11 wherein the predetermined local steady state condition is defined as variations in the fold enrichment of less than about 0.1%, or class correct less than about 7% over a span of three or more consecutive models represented on a graph.

13. The method of claim 7 wherein the training set data includes biological activity data for the compounds, wherein the biological activity data are classified by at least three different ranges of biological activity.

14. A computer readable medium comprising:
a) code for entering training set data into a digital computer, wherein the training set data are derived from a high throughput screening assay for ion channel modulators on the training set of compounds;
b) code for forming an analytical model using the training set data and a recursive partitioning process; and
c) code for selecting a subset of compounds from a test set of compounds using the analytical model.

15. The computer readable medium of claim 14 wherein the code for forming the analytical model comprises:
d) code for identifying a plurality of descriptors;
e) code for identifying a plurality of splitting points for each descriptor, each splitting point splitting each descriptor into subranges;
f) code for selecting one of the splitting points that defines a subrange that discriminates the compounds in the training set in a statistically significant manner, and
g) code for creating a recursive partitioning tree with splitting variables formed using the selected splitting points.

16. The computer readable medium of claim 15 wherein the code for forming the analytical model further comprises:
h) code for creating a plurality of recursive partitioning trees; and
i) code for selecting one of the recursive partitioning trees, wherein the selected tree is in a group of trees that collectively exhibit a predetermined local steady state condition.

17. The computer readable medium of claim 14 wherein the ion channel modulators are allosteric modulators.

18. A computer readable medium comprising:
a) code for entering training set data into a digital computer, wherein the training set data are derived from a high throughput screening assay on the training set of compounds;
b) code for forming an analytical model using a recursive partitioning process and the training set data;
c) code for selecting a subset of compounds using the analytical model; and
d) code for selecting a subset of compounds according to a predetermined pharmaceutical or therapeutic profile.

19. The computer readable medium of claim 18 wherein the code for forming an analytical model comprises:
e) code for identifying a plurality of descriptors;
f) code for identifying a plurality of splitting points for each descriptor, each splitting point splitting each descriptor into subranges;
g) code for selecting one of the splitting points that defines a subrange that discriminates the compounds in the training set in a statistically significant manner, and
h) code for creating a recursive partitioning tree having splitting variables that are formed using the selected splitting points.

20. The computer readable medium of claim 19 wherein the code for forming the analytical model further comprises:
i) code for creating a plurality of recursive partitioning trees; and
j) code for selecting one of the recursive partitioning trees, wherein the selected tree is in a group of trees that collectively exhibit a predetermined local steady state condition.
